(19) United States
(12) Patent Application Publication
(10) Pub. No.: US 2004/0143324 A1
(43) Pub. Date: Jul. 22, 2004

(54) Cosmetic eye implant, use of such implant, method for providing such implant and eyes provided therewith

(75) Inventor: Gerrit Reinold Melles, Rotterdam (NL)

Correspondence Address: BANNER & WITCOFF, LTD., 28 STATE STREET, 28th FLOOR, BOSTON, MA, 02109-9601, US

(73) Assignee: Medical Technology Transfer Holding B.V., Rotterdam (NL)

(21) Appl. No.: 10/698,965

(22) Filed: Oct. 31, 2003

(30) Foreign Application Priority Data
Dec. 24, 2002 (EP) 02080489.4

(51) Int. Cl. A61F 2/14; A61F 9/007

(52) U.S. Cl. 623/4.1; 623/905

(57) ABSTRACT

Eye implant, comprising a flat element made of biocompatible material, said element being made at least partly opaque or light reflecting, such that in use the eye implant shall be visible, once implanted in an eye.

This application claims priority from European Application Number 02080489.4, filed Dec. 24, 2002, which is hereby incorporated by reference herein.

The invention relates to an eye implant. The invention more specifically relates to a cosmetic eye implant, especially one suitable for implantation in the eye.

The invention further relates to the use of a bio compatible element as an eye implant, more specifically a cosmetic eye implant.

The invention moreover relates to a method for implanting an element in an eye as an eye implant, more specifically as a cosmetic eye implant and the results obtained therewith.

It is known to use implants in vertebrate such as a human body for both therapeutical and cosmetic purposes. The latter sort are commonly known as piercings. Piercings are normally constituted of for example stainless steel elements such as earrings and are easily releasably connected to the human body by insertion through an artificial opening in the skin, for example an earlobe or bellybutton. Part of the element stays outside the skin and is therefore visible, providing for the desired cosmetic effect.

The inventor of the present invention has had the surprising insight that implants can also be used for inter alia cosmetic purposes in or on an eye of a vertebrate. To this end an eye implant is provided according to the invention, which is characterised by the features of claim 1.

By providing a visible implant in or on the eye, especially visible in a frontal view of the eye, on or through the conjunctiva and/or the cornea, an implant is provided which will be highly visible to any body looking at or into said eye. Any encounter with a person or animal having such implant would lead almost automatically to a view of said implant. Therefore an implant according to the present invention will have a high cosmetic impact. Visible has to be understood as meaning that the implant will be noticeable from a relatively large distance, when the relevant eye is opened.

Biocompatible has to be understood as compatible for ophthalmic use, especially for use in or on an eye, without resulting in inducement of intra ocular reaction such as siderosis bulbi or medical complications in the broader sense. Opaque has to be understood as not fully transparent and/or reflective for light within the visual spectrum. Reflective has to be understood as reflecting at least part of the visual spectrum. Flat has to be understood as having a thickness in a first direction which is considerably less than the measurements perpendicular to said first direction. Cosmetic has to be understood as having substantially no medical purpose, such as a chemical or medicinal function or a vision aiding function.

It is known to use artificial lenses in the eye, for replacement of the crystalline lens which is effected by cataract surgery. Such implants are however necessarily highly transparent. It is furthermore known to use contact lenses which are temporarily positioned on the cornea, or phakic intraocular lenses that are positioned inside the eye. These also are necessarily highly transparent and must cover at least the pupil and at least part of the cornea. These known implants are therefore relatively large and not visible during use.

An eye implant according to the present invention is preferably dimensioned such, in size, form, weight and position during use that the normal eye functions are not impaired by said implant. Preferably the thickness of the element is such that during use no significant tissue deformation occurs, whereas ocular movement is not significantly hindered and the visual function of the eye is not impaired. The thickness is for example less than approximately 0.5 mm, more specifically less than 0.3 mm and preferably between 1 and 250 micron. The outer dimensions, perpendicular to said thickness for example have a maximum of approximately 12 mm, more specifically less than 6 mm and preferably between 0.5 and 4 mm.

In a further preferred embodiment an eye implant element according to the present invention is designed to be introduced into the eye, to which end the element is provided with an outer edge or periphery which is substantially rounded or otherwise smoothed and made non-cutting. Such a substantially rounded outer periphery has the advantage that after implantation the danger of for example inadvertent damage to or inflammation of the adjacent ocular tissue is prevented. Introduction of the implant into the eye has the advantage that during use no contact exists between the element and for example the eye lids, preventing the risk of impairment of the normal eye movements and other behaviour, whereas irritation of the tissue of for example the eye lids and the outer side of the conjunctiva and/or cornea is prevented. Moreover, excessive tear forming is also prevented. An other advantage is that the element is fully covered, preventing that it can for example get hooked behind artefacts of be pulled out inadvertently.

An element for an eye implant according to the present invention is preferably mainly made of one or more materials chosen from the group of platinum, platinum-iridium, titanium, nitinol, gold, silver, glass, polymethylmethacrylate, stone, jewellery stone, sand, silica, porcelain, hair, cilia, and (colored) visco elastic materials, for example hyaluronic acid. These have all been proven to be bio compatible as defined here above. Obviously also other materials, combinations of materials analoys, mixtures thereof can be used if biocompatible, that is suitable for ophthalmic use, especially intra ocular. The material or combination of materials is preferably strerilisable, such that it can either be sterilised directly prior to implantation or earlier, in which case it can be packed sterile in a container such as a box or pouch.

The present invention further relates to the use of an element as here before described as an eye implant, especially to be implanted on or in the conjunctiva, between the conjunctiva and the sclera, in the sclera or in or under the cornea.

In an element according to the present invention active elements may be used such as electronic of magnetic transponders. This may serve for example identification or locating purposes.

The present invention further discloses an eye of a vertebrate comprising an implant, especially on or in the conjunctiva, between the conjunctiva and the sclera, in the sclera, between the sclera and the cornea or in or under the cornea. Surprisingly it has been found that such element can easily be placed and will be directly visible to any body looking into said eye, for example to check the presence of the transponder. Moreover such element will not significantly impair the normal functions of the eye. Said element is preferably an element as previously disclosed. Obviously said implant can be positioned in various areas, especially in areas as disclosed in claims 19-27.

The present invention further relates to a method for implanting a cosmetic element, characterised by the features of claim 21, 24 or 26.

By using a method according to the present invention cosmetic improvements to the eye can be obtained in a relatively easy manner. Surprisingly it has been found that an element as previously disclosed can be inserted in an eye, at various levels and covered by eye tissue, without impairing the normal eye functions and without irritation to the outer layer of the conjunctiva and/or the eye lid. Especially placing such element in the conjunctiva or between the conjunctiva and the sclera could be performed by a non-medical person such as a tatooist or an optician.

In the sub claims further advantageous embodiments of products and methods according to the present invention are given.

For a better understanding of the present invention embodiments of an eye implant, use thereof, methods for implanting an element in an eye and eyes obtained therewith will be described hereafter, in relation to the accompanying drawings, in which shows:

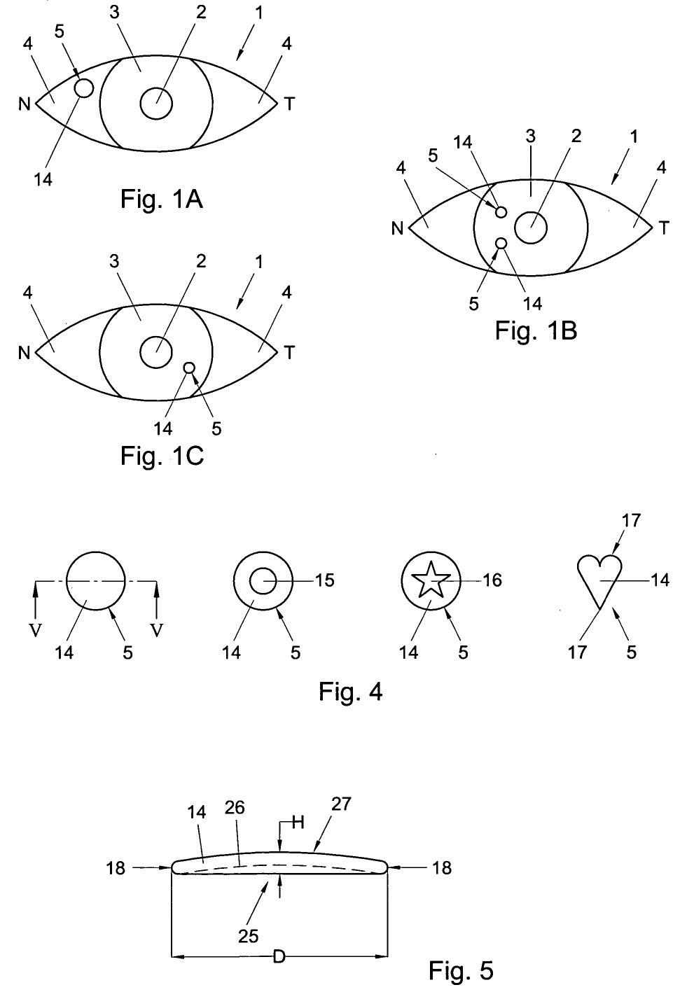

FIGS. 1A-C three embodiments of an eye according to the present invention, provided with an eye implant, visible in frontal view;

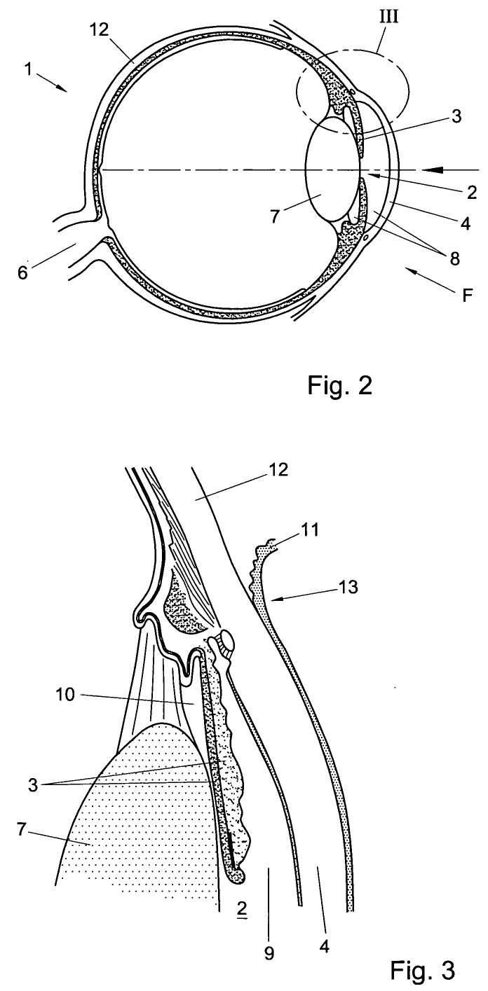

FIG. 2 in cross section schematically a human eye;

FIG. 3 in enlarged cross section part of the eye as shown in FIG. 2;

FIG. 4, schematically in frontal view a number of various shapes of eye implants according to the present invention, in enlarged views;

FIG. 5 in even further enlarged view a cross section along the line V-V in FIG. 4;

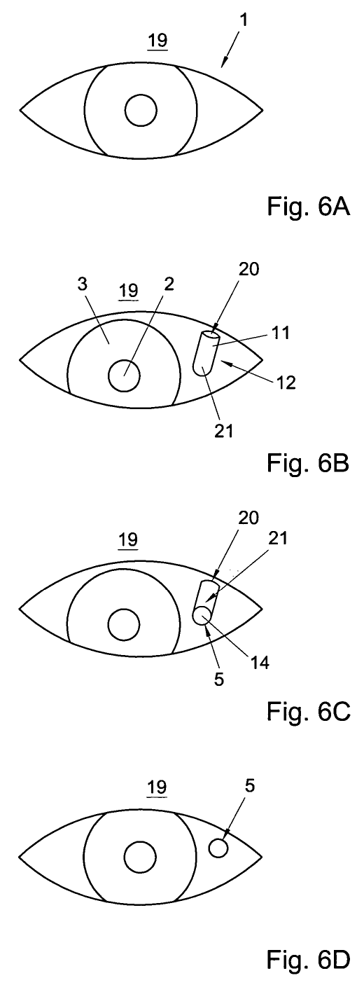

FIGS. 6A-D schematically four steps of a method according to the present invention for placing an eye implant; and

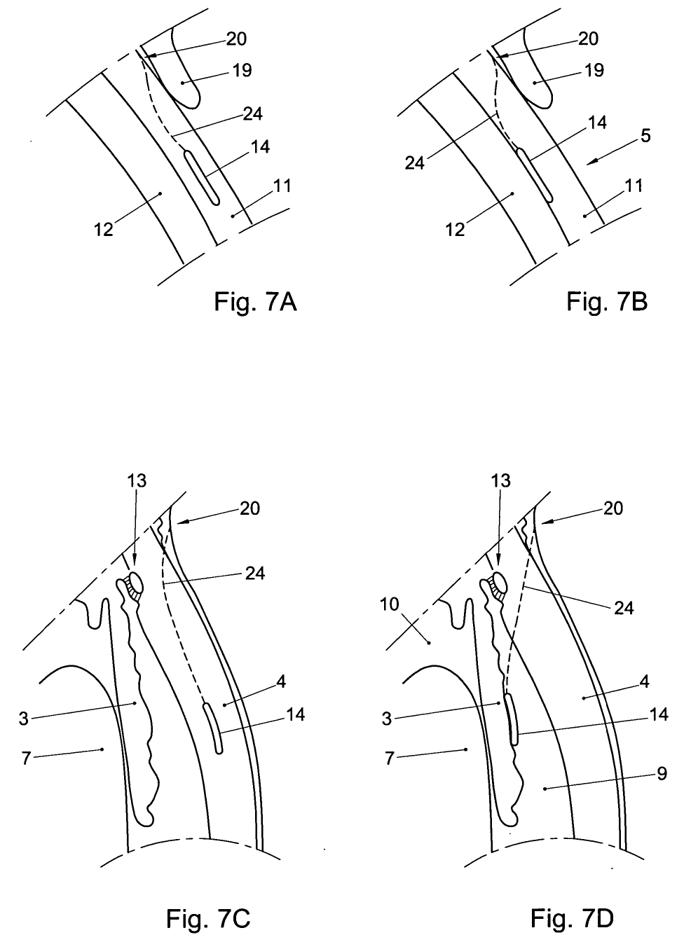

FIGS. 7A-D four different possible positions of an eye implant according to the present invention.

In this description identical or corresponding parts have identical or corresponding reference signs. In the description and drawings embodiments are given as mere examples and should in no way be understood as limiting the scope of the present invention.

In FIGS. 1A-C an eye 1 is shown, schematically, in frontal view. The eye 1 shown is a human eye but could also be the eye of another vertebrate, such as a domestic or farm animal. The eye comprises a pupil 2 with surrounding iris 3 and eye white 4 on both the temporal and nasal sides. The eye can either be a left or a right eye. In FIG. 1 the temporal side T is shown at the right side, the nasal side N at the left side.

In FIG. 1A an eye implant 5 is shown at the nasal side and spaced apart from the iris 3. In FIG. 1B two eye implants 5 are shown, in frontal view in front of the iris 3, next to the pupil 2, whereas in FIG. 1C an eye implant 5 is shown in front of the iris 3, at the lower temporal side T, spaced apart from the pupil 2. At any rate the or each implant 5 is spaced apart from the pupil, i.e. not in the pupillary axis, in order not to impair sight.

The positions shown in FIGS. 1A-C are merely examples of possible positions of eye implants 5. It will be obvious that also other positions can be possible, whereas other numbers of eye implants 5 can be positioned in the same eye. Also eye implants can be provided in the other eye of the same individual.

FIG. 2 shows a section through a human eye 1, FIG. 3 shows part of the section shown in FIG. 2, indicated by the square in FIG. 2, denominated FIG. 3. At the frontal side F of the eye 1, opposite the optic nerve 6 the pupil 2 is visible within the iris 3, behind the cornea 4. Between the cornea 4 and the iris 3, as well as between the iris 3 and the lens 7 an aqueous substance 8 is provided within the anterior chamber 9 and posterior chamber 10, respectively called aqueous humor 8. The cornea is clear and is at the limbus 13 continuous with the conjunctiva 11 and the sclera 12. The cornea 4 is more convex than the sclera 12. On each side of the iris 3, that is outside the area defined by the limbus 13, the sclera 12 extends, covered by the conjunctiva 11.

In FIG. 4 various forms and shapes of eye implants 5 are shown, basically formed by relatively flat elements 14. In FIG. 4 from left to right the element 14 is shown as a circular element 14, a ring shaped element 14, comprising an opening 15, a circular element 14, comprising a star shaped element 16 printed or otherwise provided thereon or therein and a substantially hart-shaped element 14 with rounded edges 17. These elements 14 are only shown as possible embodiments. Basically any shape and/or design is possible. An element 14 is preferably biocompatible, that is made of a suitable material to be used on or preferably in an eye. The element 14 may be made of a mono material, for example chosen from the group of platinum, platinum-iridium, titanium, gold, silver, glass, polymethylmethacrylate, stone, jewellery stone, sand, silica, porcelain, hair, cilia or can be of two or more materials, preferably chosen from the same group. Also alloys can be used or plastic. An element 14 according to the present invention is preferably made of a substantially non-transparent or opaque material but can also be made of a transparent material comprising for example light reflecting elements, inserts, prints or the like. Essential of the element 14 is that once positioned on or in an eye 1, as shown in FIGS. 1A-1C and 7A-7C the eye implant is visible in frontal view and/or light reflecting, that is reflecting light within the spectrum visible for human eyes. In alternative embodiments an element 14 according to the present invention may be made such that it is detectable by for example magnetic or electronic devices.

In FIG. 5 an element 14 is shown in enlarged cross section along the line V-V in FIG. 4. The element 14 is flat and comprises a rounded edge 18. The diameter D of the element 14 can for example be about 13 mm or less, more particularly approximately 6 mm or less. For an embodiment as shown in FIG. 1B or 1C the diameter D is preferably less than for an embodiment as shown in FIG. 1A. The diameter D is preferably within the range of approximately 0.5 and 4 mm, depending on for example the desired position within the eye 1. The thickness H, perpendicular to said diameter D is substantially less than said diameter D. The thickness H is in the embodiment shown for example less than approximately 0.5 mm, preferably between 0 and 0.3 mm. The thickness H may be as small as between 1 and 250 microns. The thickness H has to be such that the element 14 can be inserted into the eye 1, as will be discussed later referring to FIGS. 6 and 7.

The element 14 is preferably made such that it can be sterilized in a suitable manner without damage to said element.

In FIGS. 6A-D four steps are shown schematically for placing an element 14 into an eye 1, for forming an eye implant 5.

In FIG. 6A the eye 1 is prepared, for example by applying a suitable anaesthetic and antibiotic into the eye. Next the upper eye lid 19 is lifted slightly, and an opening 20 is made in at least the conjunctiva 11 by use of for example a suitable scalpel. In FIG. 6B the opening 20 is shown, made in the conjunctiva 11 in a position such that this will normally be covered by the eye lid 19. Then the conjunctiva 11 is separated partly from the underlying sclera 12 with a spatula or a (colored) visco elastic material, for example hyaluronic acid in order to form a tunnel shaped pocket 21, ending spaced apart from the pupil 2 and iris 3, for example at the same horizontal level.

Next, as is shown in FIG. 6C, the element 14 is positioned within the pocket 21, using a suitable forceps, injector or visco elastic or combination thereof, after which the opening 20 is self sealing, enclosing the element 14 for forming the eye implant 5. If at all necessary the opening 20 can be closed off by suitable sutures or any other suitable means.

Finally, the eyelid 19 is returned to the original position and the conjunctiva 11 will return back against the sclera 12, which leaves after a suitable healing period only the eye lid 5 visible in frontal view, as is shown in FIG. 6D.

In FIGS. 6A-D a method is shown for implanting an element 14, for the formation of an eye implant 5, between the sclera 12 and the conjunctiva 11, as shown in FIG. 7B, spaced apart from the iris 3, as is shown in FIG. 1A. However, according to the present invention the eye implant 5 can also be provided by inserting the element 14 in a different manner, for example within the conjunctiva 11, as is shown in FIG. 7A, within the cornea, in front of the iris 3, as is shown in FIG. 7C and FIG. 1B, or on the iris 3, within the interior chamber 9, behind the transparent cornea 4, as is shown in FIGS. 1C and 7D. In FIGS. 7A-7D the incisions made for placing the element 14 in the respective positions are shown by dotted lines 24, extending between the opening 20 and the element 14.

An element 14 can be made slightly spherical or be provided with a concave recess 25, as schematically shown in FIG. 5 by the dotted line 26, such that it will connect to the relevant part of the eye 1 more suitably. Also attachment means can be provided (not shown) for connecting said element to eye tissue, for example the iris 13 in a position as shown in FIG. 7D. Such attachment means can for example be small hooks, claws, glue, pins or the like. The surface 27 of an element 14 which is during use visible may be provided with a print or the like, for example glittering for extra visual effects.

Although in the embodiments shown the implant 5 is provided within the eye 1, that is embedded in eye tissue, it may also be provided at least partly on the outside of the eye, that is on or partly in the conjunctiva and/or the sclera and/or the cornea. In such embodiment it is especially preferred to provide said attachment means in order to prevent migration of said element over the eye, which might impair the normal functions of the eye, for example vision.

One or more elements 14 for forming an eye implant 5 may be sterilized and packed in a container, for example a pouch, sealed against influence from the environment. A suitable instruction may be packed in or with said container, such that the intended use of said element is clear. A method according to the present invention, especially for positioning the element 14 as is shown in FIGS. 7A and 7B and possibly 7C may be performed by any suitable person, for example a tatooist an optician or a surgeon. The eye implant 5 is basically of a cosmetic nature, visible in frontal view and may be reflective for visible light and/or light emitting.

The lower temporal or nasal area provides for a high exposure of the cosmetic implant. The implant is preferably positioned spaced apart from the pupillary axis and placed such that it does not interfere with for example the film of tear-fluid on the cornea.

The visibility of the implant can be provided for or enhanced by for example color, fluorescence, use of different materials, surface finish, prints and combinations thereof.

The implant may also consist of multiple particles, or multiple particles evenly distributed within a carrier substance like a visco elastic, for example hyaluronic acid, that are injected with the carrier into the eye tissue. The particles are non-transparent, for example glittering particles.

The invention is by no means limited to the embodiments as shown and described. Many variations thereof are possible within the scope of the invention.

For example an element according to the present invention may have other forms or dimensions, and may be made of other materials suitable for ophthalmic use. When more than one element 14 is positioned in the same eye, these elements may well be interconnected. Openings 20 and incisions or tunnels 21 may be made in other positions and with other means, extending in different directions.

1. Eye implant, comprising a flat element made of biocompatible material, said element being made at least partly opaque or light reflecting, such that in use the eye implant shall be visible, once implanted in an eye.

2. Eye implant according to claim 1, wherein said element is substantially opaque and made light reflecting.

3. Eye implant according to claim 1, wherein said element is substantially disc shaped.

4. Eye Implant according to claim 1, wherein the thickness of said flat element is less than approximately 0.5 mm, preferably between 0 and 0.3 mm and most preferably between 1 and 250 micron.

5. Eye implant according to claim 1, wherein said element has a relatively small thickness and dimensions perpendicular to said thickness which are less than 12 mm, more particularly less than 5 mm and preferably between 0.5 and 4 mm.

6. Eye implant according to claim 1, wherein the implant has an outer edge which is substantially rounded.

7. Eye implant according to claim 1, wherein the implant is a cosmetic implant.

8. Eye implant according to claim 1, wherein the implant is substantially made of a material or combination of materials chosen from the group of platinum, platinum-iridium, titanium, gold, silver, glass, polymethylmethacrylate, stone, jewellery stone, sand, silica, porcelain, hair, cilia.

9. Eye implant according to claim 1, wherein the material or combination of materials used allows sterilisation.

10. Eye implant according to claim 1, wherein de implant has at least one concave surface, preferably one concave and one convex surface.

11. Eye implant according to claim 1, enclosed in a closed container like a box or pouch, said eye implant being sterilised or sterilisable within said container.

12. Eye implant according to claim 1, wherein attachment means are provided for permanently or releasably connecting said implant to an eye, especially to the conjunctiva, sclera, cornea or iris of an eye.

13. Eye implant according to claim 1, positioned on an eye, especially on the conjunctiva of an eye, visible in a frontal view of said eye with open eyelids.

14. Eye implant according to claim 1, positioned in an eye, especially in the conjunctiva of an eye, visible in a frontal view of said eye with open eyelids.

15. Eye implant according to claim 1, positioned in an eye, especially between the conjunctiva and the sclera of an eye, visible in a frontal view of said eye with open eyelids.

16. Eye implant according to claim 1, positioned in an eye, especially on or in the sclera of an eye, visible in a frontal view of said eye with open eyelids.

17. Eye implant according to claim 1, positioned in an eye, especially in or on the cornea or iris of an eye, visible in a frontal view of said eye with open eyelids.

18. Eye implant according to claim 1, wherein the implant consists of or comprises multiple particles or multiple particles evenly distributed within a carrier substance, for example a visco elastic material, for example hyaluronic acid.

19. Use of a flat, bio compatible element, especially according to claim 1 as a cosmetic eye implant.

20. Eye of a vertebrate, comprising a cosmetic eye implant positioned in, on or between the conjunctiva, sclera, cornea and/or iris thereof.

21. Eye according to claim 20, wherein said eye implant is positioned spaced apart from the pupil of said eye and is visible from a frontal view.

22. Method for implanting an element in an eye, comprising the steps of:
providing said element made of eye compatible material, which element is at least partly opaque and/or light reflecting;
providing an opening in the conjunctiva of an eye;
dissecting part of the conjunctiva from the conjunctiva or sclera through said opening;
inserting said element in the conjunctiva or between the conjunctiva and the sclera through said opening; and
closing said opening preferably by self sealing, thereby enclosing said element

23. Method according to claim 22, wherein said opening is provided spaced apart from a desired implantation site, whereby a subconjunctive tunnel is provided from said opening to said implantation site, the opening preferably being closed with self absorbing suture, glue or the like.

24. Method according to claim 22, wherein an implantation site is used situated in a temporal or nasal conjunctive area, preferably a lower temporal or nasal area, about 2 to 5 mm spaced apart from the relevant part of the limbus.

25. Method for implanting an element in an eye, comprising the steps of:
providing said element made of eye compatible material, which element is at least partly opaque and/or light reflecting;
providing an opening in the conjunctiva of an eye;
providing a scleral tunnel into the clear cornea of said eye, connected to said opening;
introducing said element into said scleral tunnel through said opening into the peripheral cornea; and
closing said opening, thereby enclosing said element in said scleral tunnel.

26. Method according to claim 25, wherein an implantation site is used situated in a temporal or nasal cornea area, preferably a lower temporal or nasal area, about 1 to 2 mm spaced apart from the relevant part of the limbus.

27. Method for implanting an element in an eye, comprising the steps of:
providing said element made of eye compatible material, which element is at least partly opaque and/or light reflecting;
providing an opening in the conjunctiva of an eye;
providing a scleral incision into the clear cornea of said eye, connected to said opening;
providing an incision from said scleral incision into the anterior chamber of said eye;
introducing said element through said opening and said incisions into said anterior chamber and positioning said element on the iris of said eye; and
closing said opening.

28. Method according to claim 27, wherein an implantation site is used situated in an interpalpebral area of the mid iris or of the mid-central iris.

29. Set of an eye implant according to claim 1, at least one scalpel or similar knife, visco elastic material and at least one forceps.
